(19) United States
(12) Patent Application Publication
(10) Pub. No.: US 2004/0042073 A1
(43) Pub. Date: Mar. 4, 2004

(54) Intraocular lenses provided with angled edges to prevent posterior capsular opacification

(76) Inventor: Joel Pynson, Toulouse (FR)

Correspondence Address: YOUNG & THOMPSON, 745 SOUTH 23RD STREET 2ND FLOOR, ARLINGTON, VA, 22202

(21) Appl. No.: 10/432,903

(22) PCT Filed: Oct. 31, 2002
(86) PCT No.: PCT/FR02/03754

(30) Foreign Application Priority Data
Oct. 31, 2001 (FR) 01 14100

(51) Int. Cl. A61B 1/00; G02B 23/00

(52) U.S. Cl. 359/367

(57) ABSTRACT

An intraoluclar refractive lens comprises an optic portion (34) with an outer peripheral edge (36) and one or more, preferably two, three or four equidistant haptic elements (38). Each haptic element (38) has the same shape, with a trapezoid or rhomboid cross-section. The edge of the outer rear surface of each haptic element (38) and the edge of the rear optic element are shaped like acute angles to prevent posterior capsular opacification.

The present invention relates to intraocular lenses (IOL) and a method to produce and use these latter. More particularly, the present invention relates to IOLs with angular edges to prevent posterior capsular opacification in aphaic eyes whose natural lens of the eye has been surgically extracted.

The extraction of the cataract forms a portion of operations most usually carried out in the United States and throughout the world. The crystalline lens is located within a so-called capsular sac or crystalline lens capsule located in the posterior chamber of the eye. To have access to a crystalline lens with cataracts, the usual way to proceed is by making an incision in the edge of the eye so as to introduce a surgical instrument into the anterior chamber of the eye. In the case of extraction of an extracapsular cataract, there is followed a capsulo-hexis procedure in which a portion of an anterior membrane of the capsule of the crystalline lens adjacent the iris of the eye is extracted with the help of a cutting surgical instrument so as to provide direct access to the crystalline lens affected by cataracts, from the anterior chamber. The crystalline lens affected with cataracts is thus extracted with the help of various known methods, including phaco-emulsification. Phaco-emulsification is a procedure which involves the application of ultrasonic energy to the crystalline lens having cataracts, so as to shatter the crystalline lens into small pieces which can be sucked out of the crystalline lens capsule. Except for the portion of the anterior membrane of the capsule of the crystalline lens extracted during the capsulo-hexis procedure, the capsule of the crystalline lens remains essentially intact during all the duration of an extraction of the extracapsular cataract. Involving extraction of the crystalline lens having cataracts, an artificial intraocular lens (IOL) insert, as described in greater detail below, is implanted in the usual way within the crystalline lens capsule so as to irritate the refractive function of the extracted natural crystalline lens.

For years, IOL implants have been used in aphaic eyes in which the natural crystalline lens of the eye has been removed. Numerous different designs of IOL have been used for years past and have been used with success in aphaic eyes. At present, the designs of successful IOLs comprise principally an optical portion with its supports, called haptics, connected to and surrounding at least a portion of the optical portion. The haptic portions of an IOL are designed to support the optical portion of the IOL in the crystalline lens capsule, the anterior chamber or the posterior chamber of an eye.

IOLs having commercial success have been produced from a variety of biocompatible materials, ranging from more rigid materials such as polymethylmethacrylate (PMMA) to more flexible materials capable of being bent or compressed such as silicones and certain acrylics. Haptic portions of the IOLs have been formed separately from the optical portion and then later connected to this portion by processes such as heat, physical stapling and/or chemical cementing. The IOLs with haptics attached in this manner are commonly called “multi-piece” IOLs. The IOLs are also commonly produced with haptics formed like an integral portion of the optical portion in what is commonly called “monoblock” IOLs.

The softest most flexible IOLs have had a popularity that has increased during recent years because of their ability to be compressed, bent, rolled or to withstand other deformations. These softer IOLs can be deformed before their insertion through an incision in the cornea of the eye. Following insertion of the IOL in the eye, the IOL resumes its original shape before deformation because of the memory characteristics of the soft material.

Softer more supple IOLs such as those that have been described, can be implanted in an eye through an incision which is much smaller, which is to say 2.8 to 3.2 mm, than that necessary for more rigid IOLs, which is to say 4.8 to 6.0 mm. A larger incision is necessary for the more rigid IOLs, because the lens must be inserted through an incision in the cornea slightly greater than that of the diameter of the optical portion of the rigid IOL. As a result, the more rigid IOLs have become less popular on the market because of the need for larger incisions which are associated with increased incidence of post-operative complications such as induced astigmatism.

Although the extraction of the portion having cataracts, with replacement by IOL implant, offers considerable advantages to most patients having a cataract, this is not always the case. It is estimated that up to thirty percent (30%) of all the patients which receive an IOL implant within the crystalline lens capsule of the eye, thereafter develop posterior capsular opacification (PCO) or secondary characteristics, during the five years following the surgical intervention. The PCO is an opacification of the IOL implants caused by the deposition of cells and fibers on the posterior surface of the implant of the IOL and on the posterior capsular membrane. These deposits of cells and fibers obstruct the passage of light through the implant of the IOL and obscure the vision of the patient. The principal cause of PCO is the migration and the proliferation of the residual epithelial cells of the lens on the capsular membrane.

Because of the insufficiencies observed in the past and present designs of IOLs, there exists a need for IOL implants designed to prevent PCO.

The intraocular lenses (IOLs) produced according to the present invention have an optical portion having an external peripheral edge and two or more, but preferably two, three or four equidistant haptic elements to support the optical portion in the eye of the patient. Preferably, each of the haptic elements is of the same shape to obtain a helical effect to facilitate implantation, the rotation and centering of the IOL. The helical effect also improves the accessibility to the interior of the capsule to clean the cortical remains. Each of the haptic elements has an internal portion and an external portion. The internal portion of each haptic element comprises an enlarged securement portion connected permanently to the external peripheral edge of the optical portion. The external portion of each haptic element comprises a free rounded end. Each haptic element comprises an elongated central portion in the form of an arc which extends between the enlarged securement portion and the free rounded end. From the enlarged securement portion and all along the central elongated portion in the form of an arc, the dimensions of the haptic element can be constant or can vary. The particular angular shape of the haptic elements permits rotation of the IOL and prevents posterior capsular opacification.

As a result, an object of the present invention is to provide intraocular lenses to be used in aphaic eyes.

Another object of the present invention is to provide intraocular lenses to be used in aphaic eyes capable of being implanted through a small incision.

Another object of the present invention is to provide intraocular lenses which prevent posterior capsular opacification.

Another object of the present invention is to provide intraocular lenses which permit improved facility of implantation, of rotation and of centering of said lenses.

Another object of the present invention is to provide intraocular lenses which are biocompatible to be used in aphaic eyes.

Still another object of the present invention is to provide intraocular lenses which are resistant to decentering within the eyes.

These and other objects and advantages according to the present invention, of which several will be described in a specific manner and others not, will become clear from the detailed description, from the drawings and the claims hereinafter, in which the same characteristics are designated by the same reference numerals.

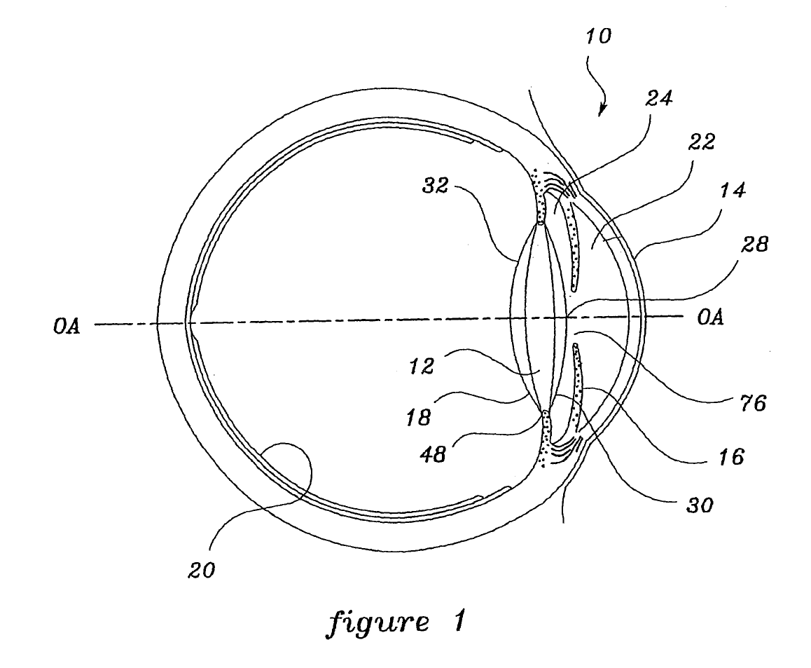

FIG. 1 is a schematic representation of the interior of a human eye comprising a natural crystalline lens and a refringent IOL implanted in the capsule of the crystalline lens of the eye;

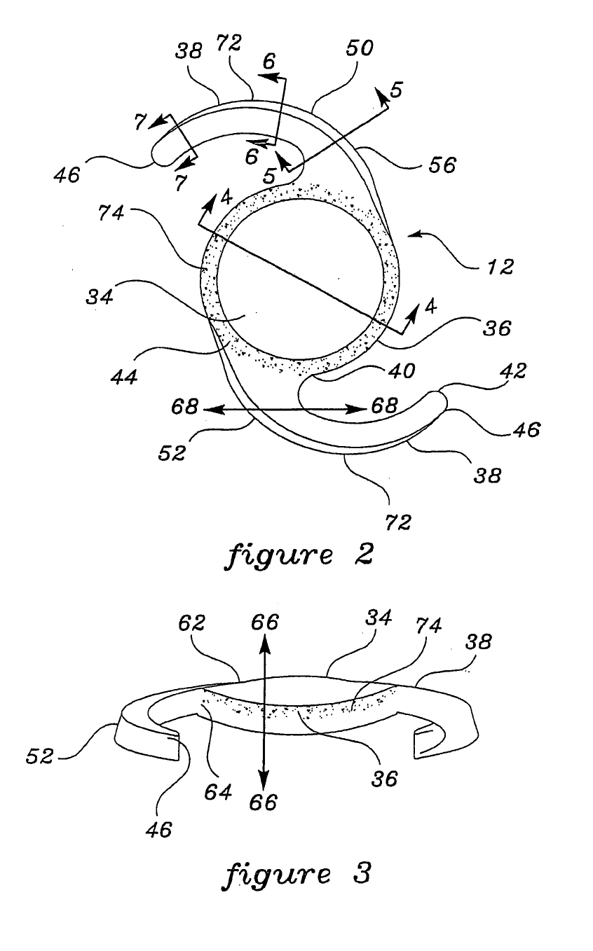

FIG. 2 is a plan view of an IOL with two haptics, produced according to the present invention;

FIG. 3 is a side view of the IOL of FIG. 2;

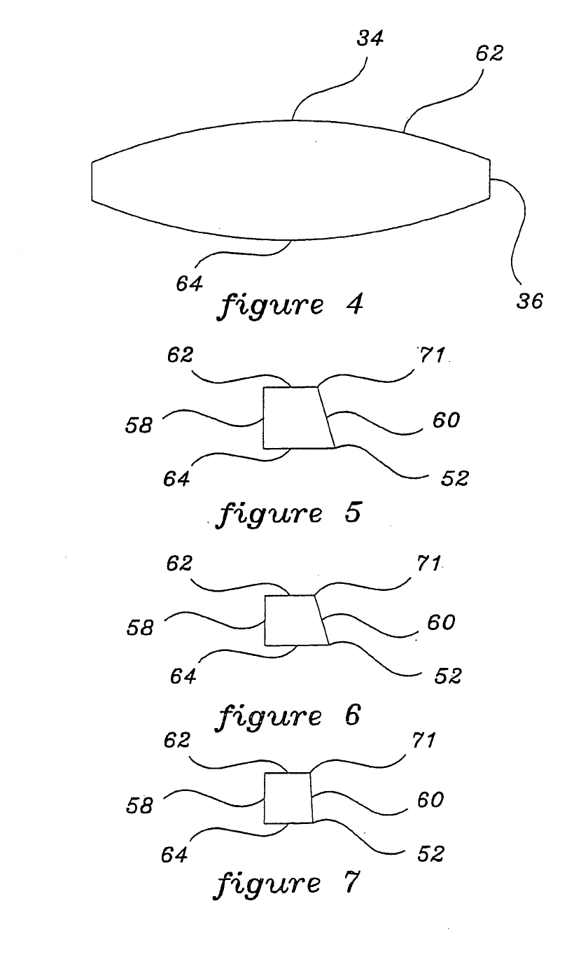

FIG. 4 is a transverse cross sectional view of an optical portion of the IOL of FIG. 2, taken along the line 4-4;

FIG. 5 is a transverse cross sectional view of a haptic element of the IOL of FIG. 2, taken along the line 5-5;

FIG. 6 is a transverse view of a haptic element of the IOL of FIG. 2, taken along the line 6-6;

FIG. 7 is a transverse view of a haptic element of the IOL of FIG. 2, taken along the line 7-7;

FIG. 8 is a plan view of another embodiment of an IOL with two haptics, produced according to the present invention;

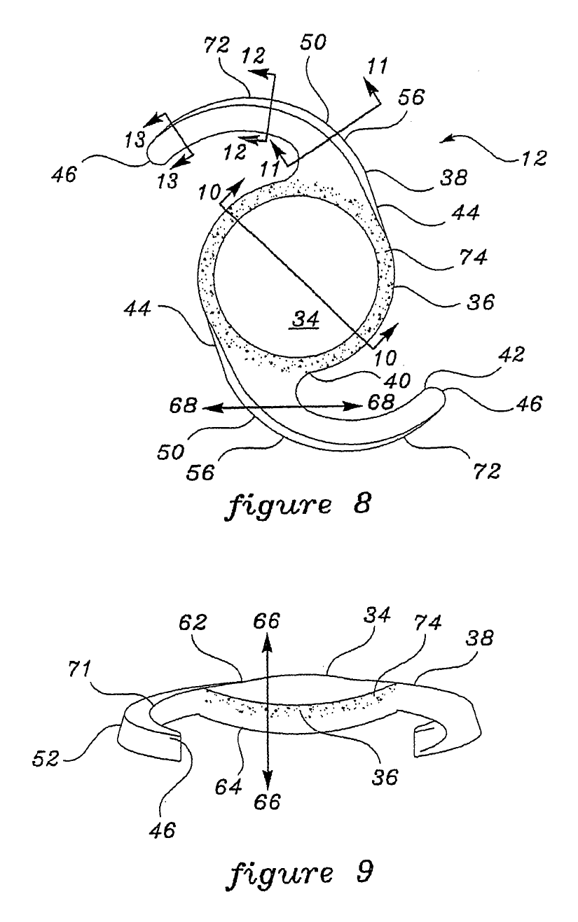

FIG. 9 is a side view of the IOL of FIG. 8;

FIG. 10 is a transverse cross sectional view of an optical portion of the IOL of FIG. 8, taken along the line 1010;

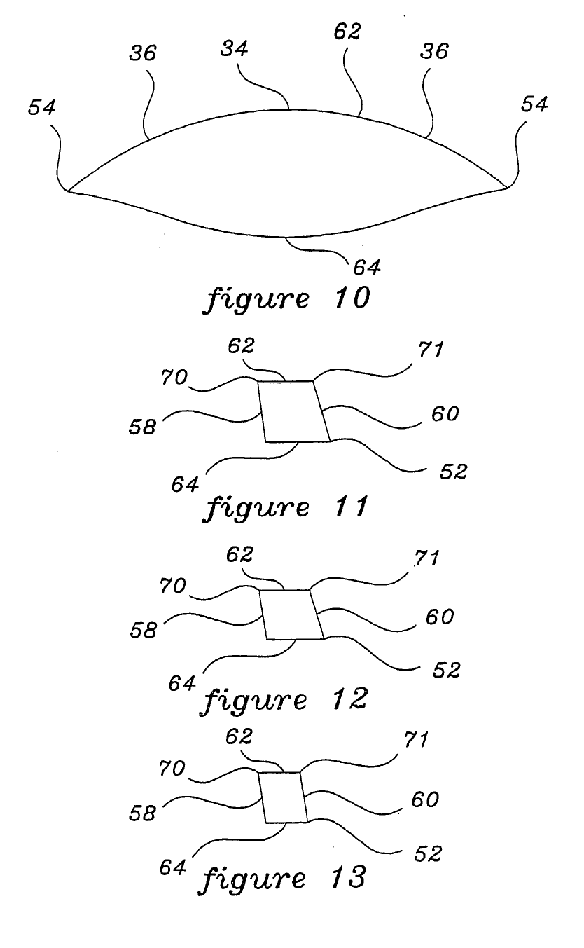

FIG. 11 is a transverse cross sectional view of a haptic element of the IOL of FIG. 8, taken along the line 1111;

FIG. 12 is a transverse cross sectional view of a haptic element of the IOL of FIG. 8, taken along the line 1212;

FIG. 13 is a transverse cross sectional view of a haptic element of the IOL of FIG. 8, taken along the line 1313;

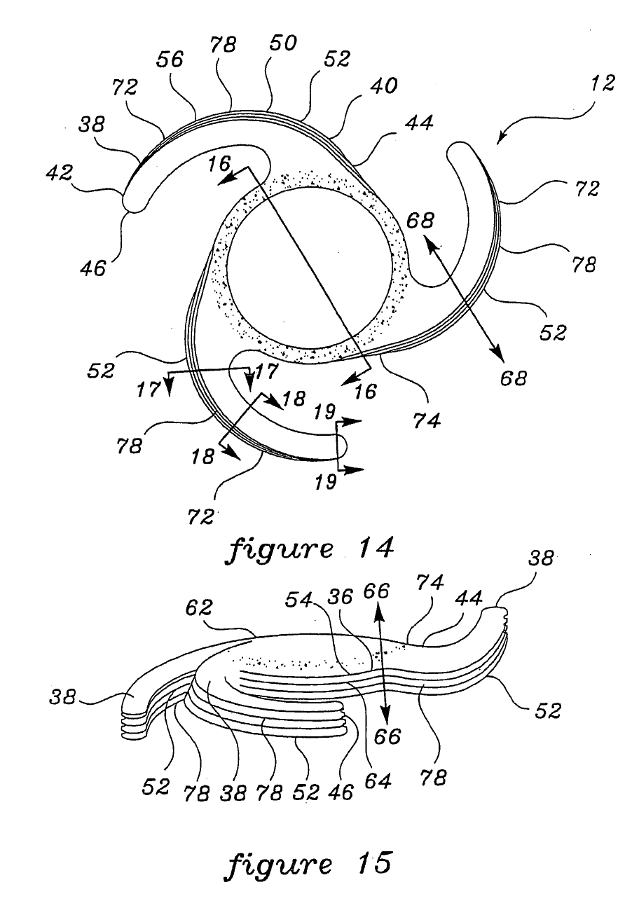

FIG. 14 is a plan view of another embodiment of an IOL with three haptics produced according to the present invention;

FIG. 15 is a side view of the IOL of FIG. 14;

FIG. 16 is a transverse cross sectional view of an optical portion of the IOL of FIG. 14, taken along the line 1616;

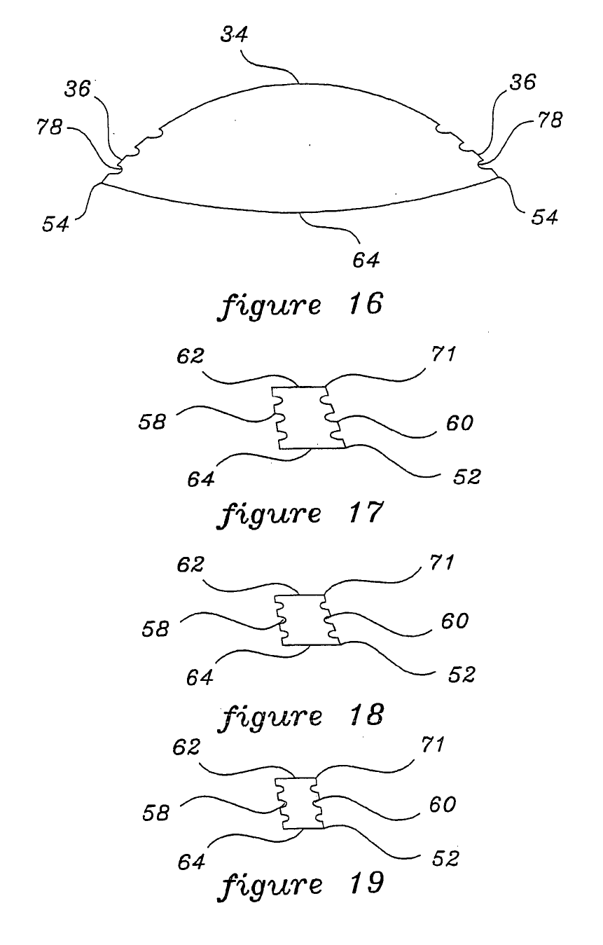

FIG. 17 is a transverse cross sectional view of a haptic element of the IOL of FIG. 14, taken along the line 1717;

FIG. 18 is a transverse cross sectional view of a haptic element of the IOL of FIG. 14, taken along the line 1818; and

FIG. 19 is a transverse cross sectional view of a haptic element of the IOL of FIG. 14, taken along the line 1919.

FIG. 1 is a schematic view of an eye 10 showing the structures which are related to the implantation of intraocular lenses (IOLs) 12 according to the present invention. The eye 10 comprises an optically clear cornea 14 and an iris 16. A crystalline lens capsule 18 and a retina 20 are disposed behind the iris 16 of the eye 10. The eye 10 also comprises an anterior chamber 22 located in front of the iris 16 and a posterior chamber 24 located between the iris 16 and the crystalline lens capsule 18. The IOL 12 is preferably implanted in the crystalline lens capsule 18 of the aphaic eye. When they are used in aphaic eyes, IOLs 12 serve to replace the ailing natural crystalline lenses removed surgically, for example following a cataract operation. The eye 10 also comprises an optical axis OA-OA which is an imaginary line passing through the optical center 28 of the anterior surface 30 and of the posterior surface 32 of the crystalline lens capsule 18. The optical axis OA-OA in the human eye 10 is generally perpendicular to a portion of the cornea 14, to the crystalline lens capsule 18, and to the retina 20.

The IOLs according to the present invention, shown in FIGS. 2, 8 and 14, and generally designated by the reference numeral 12, are designed to be implanted preferably in the crystalline lens capsule 18 of an aphaic eye. The IOL 12 has an optical portion 34 with an external peripheral edge 36. Two or more, but preferably two, three or four, equidistant haptic elements 38 are formed preferably integrally with the peripheral edge 36 of the optical portion 34. Each haptic element 38 has a similar shape to obtain a helical appearance to facilitate the rotation and centering of the IOL 12 during implantation within the eye 10 and to obtain the rotation of the IOL 12 as described in greater detail below.

Each haptic element 38 is produced so as to have an internal portion 40 and an external portion 42. The internal portion 40 of each haptic element 38 comprises an enlarged securement portion 44 preferably formed integrally and connected permanently with the external peripheral edge 36 of the optical portion 34. As a modification, however, the enlarged securement portion 44 of each haptic element 38 can be fixed permanently to the optical portion 34 by stapling, chemical polymerization or by any other methods known to those skilled in the art. Each haptic element 38 also comprises on the external portion 42 a free rounded end 46 designed to avoid contact with internal surfaces 48 of the crystalline lens capsule 18 of the aphaic eye 10.

The haptic elements 38 are formed with elongated central portions of arcuate shape 50 which extend between the enlarged securement portions 44 and the free ends 46. As shown best in FIGS. 5-7, 11-13 and 17-19, from the enlarged securement portion 44 and all along the central elongated portion 50 in the form of an arc, the transverse section of the haptic element 38 has a trapezoidal shape or, as a modification, rhomboidal, to obtain the desired characteristics for prevention of PCO.

As is seen in FIGS. 5 to 7, the cross section of the haptic element 38 on the lines 5/5, 6/6 and 7/7 is trapezoidal, the external side 60 of the trapeze comprising at its lower portion an acute angle 52 and at its upper portion an obtuse angle 71.

The particular shape of the haptic elements 38 produces two effects: on the one hand, thanks to the acute haptic edge 52, the migration of the capsular cells along the posterior surface 32 is blocked, and on the other hand, thanks to the obtuse external anterior haptic edge 71, the rapid obturation between the anterior surface 30 and the posterior surface 32 is possible. This obturation arises from the phenomenon known under the name of symphysis, according to which, when the capsular sac has been emptied, the two walls 30 and 32 of said sac bond to each other. When they are thus bonded together, there is no more migration of the capsular cells.

Preferably, the acute angle 52 is comprised between 10 and 45°.

The haptic element 38 can be stable or its dimensions can vary from the enlarged securement portion 44 and all along the central portion 50 in the plane 68-68 substantially perpendicular to the optical axis OA-OA. The haptic elements 38 with regular dimensions from the enlarged securement portion 44 and all along the central portion 50, can be illustrated best by FIGS. 6, 12 and 18 and by the dimensions which are provided thereon. However, because of the increasing complexity of the embodiments having variable dimensions, they are treated more in detail below.

In the modes of production with haptic elements whose dimensions vary, from the enlarged securement portion 44 to the free rounded end 46, the width of the haptic element 38 gradually decreases in the plane 68-68. Roughly at the enlarged securement portion 44, the anterior surface 62 has a width of about 1.2 to 1.4 mm. The anterior surface 62 is preferably more narrow by about 30 percent, than the posterior surface 64, which has preferably a width of about 1.6 to 1.9 mm, as shown by the embodiments of FIGS. 5, 11 and 17. As shown by the embodiments of FIGS. 6, 12 and 18, at about the mid-section 56, the anterior surface 62 has a width of about 1.0 to 1.2 mm. The anterior surface 62 is preferably more narrow by about 15%, than the posterior surface 64, which preferably has a width of about 1.2 to 1.5 mm. As shown by the embodiments of FIGS. 7, 13 and 19, roughly toward the free rounded end 46, the anterior surface 62 and the posterior surface 64 have about the same width of about 0.5 to 1.0 mm.

Proceeding from the enlarged securement portion 44 toward the free rounded end 46, the haptic element 38 has a uniform thickness all along the plane 66-66 parallel to the optical axis OA-OA. As shown by the embodiments of FIGS. 5-7 and 17-19, the surface of the haptic internal edge 58 has a thickness of about 0.20 to 0.80 mm but preferably of about 0.34 to 0.48 mm. As shown by the embodiments of FIGS. 11-13, the surface of the haptic internal edge 58 can, as a modification, be given any angular inclination relative to the anterior surface of the IOL 62 and to the posterior surface of the IOL 64, to form an acute internal anterior haptic edge 79 along the anterior surface of the IOL 62. The surface of the haptic external edge 60, as shown by the embodiments of FIGS. 5-7, 11-13 and 17-19, is angular rather than perpendicular relative to the anterior surface of the IOL 62 and to the posterior surface of the IOL 64 to form an acute external posterior haptic edge 52 along the posterior surface of the IOL 64.

Generally speaking, when moving from the enlarged securement portion 44 toward the end portions 42, the width of the haptic elements 38 can decrease in the plane 68-68 whilst the thickness in the plane 66-66 remains the same.

The IOL 12 is preferably produced with an optical portion 34 having a diameter of about 4.5 to 9.0 mm, but preferably about 5.0 to 6.0 mm and more precisely of 6.0 mm and a thickness of about 0.2 mm to 1.0 mm, but preferably about 0.2 to 0.8 mm and more precisely from 0.3 to 0.5 mm on the peripheral edge 36, as shown by the embodiment of FIG. 2. As a modification, as shown in the embodiments of FIGS. 8 and 14, the peripheral edge 36 of the optical portion 34 is angular rather than perpendicular relative to the anterior surface of the IOL 62 and to the posterior surface of the IOL 64. By thus angulating the peripheral edge 36, there is formed an acute posterior optical edge 54 along the posterior surface of the IOL, as shown best in FIGS. 10 and 16.

As can be seen in FIG. 10, the peripheral edge 36 is disposed in prolongation of the surface of the optical portion 34.

The haptic elements 38 extend from the optical portion 34 in the form of an arc generally speaking, and their length increases or decreases as a function of the diameter of the optical portion 34. When the diameter of the optical portion 34 increases, the overall length of the haptic elements 38 decreases. Similarly, when the diameter of the optical portion 34 decreases, the overall length of the haptic elements 38 increases. Generally speaking, the haptic elements are formed to have a length of about 2.6 to 6.0 mm, but preferably between about 3.4 to 5.5 mm, and more precisely about 4.8 mm, measured between the center of the enlarged securement portion 44 and the center of the rounded free end 46. The IOL measures preferably between 11 and 13 mm total, between the contact zone 72 and the opposite contact zone 72.

During implantation, the IOL 12 is preferably positioned with the contact zones 72 of the external posterior haptic edges 52 in contact with the internal surfaces 48 of the capsule of the crystalline lens 18. The haptic elements 38 of the IOL 12 are inclined outwardly to be in continuous contact between the contact zones 72 and the internal surfaces 48. In thus positioning the IOL 12 within the crystalline lens capsule 18, the central portions 50 of the haptic elements 38 are slightly deflected inwardly in the plane 68-68. The central portions 50 thus deflect under the forces of compression because they have a reduced width relative to that of the enlarged securement portions 44. During flexure of the haptic elements 38 to the central portions 50, the surfaces of the internal haptic edge 58 move closer to the external peripheral edge 36. During implantation, the external posterior haptic edges 52, preferably with the posterior optical edge 54 of the IOL 12, come into contact with the internal surfaces 48 of the crystalline lens capsule 18 to prevent PCO. The external posterior haptic edges 52 and the posterior optical edge 54 prevent PCO by serving as a barrier to the migration and the cellular proliferation within the crystalline lens 18. As a result, when the IOL 12 is used as a refringent lens, there is obtained a refringent correction that is stable and reliable.

The suitable materials for the production of IOLs 12 comprise, without thereby being limited, pliable or compressible materials such as silicone polymers, hydrocarbon and fluorinated hydrocarbon polymers, soft acrylic polymers without a water content, with a low water content and with a high water content, polyesters, polyamides, polyurethane, silicone polymers with hydrophilic monomer additions, polysiloxane elastomers containing fluorine and their combinations. The preferred material for the production of IOL 12 according to the present invention is a hydrophilic or hydrophobic acrylic material such as those known to persons skilled in the art. The poly(hydroxyethyl methacrylate-co-hydroxyhexyl methacrylate) (poly(HEMA-co-HOHEXMA) and methyl methacrylate-hydroxyethyl methacrylate (MMA-HEMA) are the preferred hydrophilic acrylic materials used for the production of IOLs 12 because of their equilibrium water content comprised between about 17 and about 27 percent by weight and of a high refractive index of about 1.46 or more, which is higher than that of the aqueous humor of the eye, which is to say 1.33. A high index of refraction is a desirable characteristic for the production of an IOL to give a high optical power with minimum optical thickness. By using the material having a high index of refraction, the defects of visual acuity can be corrected with the help of a thinner IOL. Poly(HEMA-co-HOHEXMA) and MMA-HEMA are also desirable materials for the production of IOL 12 because of their mechanical resistance which is suitable to resist considerable physical manipulation. Poly(HEMA-co-HOHEXMA) and MMA-HEMA also have desirable memory properties suitable for use as IOLs. IOLs produced from a material having good memory properties such as those of poly(HEMA-co-HOHEXMA) and MMA-HEMA peel off an eye in a controlled manner rather than explosively, to take their predetermined shape. Explosive and peeling of an IOL is not desirable because of the potential damage to the delicate tissues within the eye. Poly(HEMA-co-HOHEXMA) and MMAHEMA also have indeformability in the eye.

Similarly, the IOLs 12 can be produced with a variety of materials having various physical characteristics. For example, IOL 12 can be produced to have an optical portion 34 made of a hydrophilic acrylic material with a high index of refraction, the haptic elements 38 made of a more rigid material than that of the optical portion 34, and contact zones 72 made of the same material as that of the optical portion 34 or of another material having a lower index of refraction and a higher glass transition temperature.

Although the teachings of the present invention will preferably be applied to soft or pliable IOLs made of a pliable or compressible material, the same is true for harder lenses, less flexible, made of a relatively rigid material such as polymethyl methacrylate (PMMA) having flexible haptics made either of the same material or of a different material.

The optical portion 34 of IOL 12 can be a lens with a positive power between 0 and about +40 diopters or a lens with a negative power between 0 and about −30 diopters. The optical portion 34 can be biconvex, plano-convex, plano-concave, biconcave or concavo-convex (meniscal) as a function of the necessary power to obtain the central and peripheral thickness for effective manipulation.

As an option, the optical portion 34 of the IOL 12 can be formed with a zone 74 of reduced brightness having a width of about 0.25 to 0.75 mm but preferably about 0.3 to 0.6 mm and more preferably 0.5 mm adjacent the external peripheral edge 36 to reduce the brightness when the external peripheral edge 36 of the IOL 12 is struck by light which penetrates the eye 10 in the case of high luminosity or at other times when the pupil 76 is dilated. The zone of reduced brightness 74 is characteristically made of the same material as that of the optical portion 34, but it can be opaque, colored or designed in a conventional manner to block or diffuse the light in the plane having the optical axis OA-OA.

IOL 12 is preferably produced by first producing disks of a single or several materials from one or several materials selected as described in U.S. Pat. Nos. 5,217,491 and 5,326,506. IOL 12 can thus be machined from disks of material in a conventional manner. Once machined, IOL 12 can be polished, cleaned, sterilized and packaged with a conventional process known to those skilled in the art.

IOL 12 is used in the eye 10 by providing an incision in the cornea 14 and by inserting the IOL 12 into the posterior chamber 24 and by closing the incision according to methods known to those skilled in the art. However, IOL 12 can preferably be used in the eye 10 by providing an incision in the cornea 14 and the crystalline lens capsule 18, by extracting the natural crystalline lens, by inserting the IOL 12 into the crystalline lens capsule 18 and by closing the incision according to methods known to those skilled in the art.

IOL 12 according to the present invention provides a refringent lens suitable for use in the crystalline lens capsule 18 or in the posterior chamber 24, but preferably in the capsule of the crystalline lens 18 by reason of its characteristics for preventing PCO. IOL 12 has haptic elements 38 of similar shape to minimize or limit the decentering of IOL 12 and the distortion of vision. The similar shape of the haptic elements 38 permits in the same manner the rotation of the IOL 12 for better positioning and better adjustment to the interior of the crystalline lens capsule 18. This better adjustment within the crystalline lens capsule 18 has an advantage because one or several sizes of lenses will adjust in a suitable manner to most of the dimensions of the eye 10. By providing a “universal” lens such as that according to the present invention, the clinical risks for the patients are minimized that are caused by poorly dimensioned lenses. Similarly, there is eliminated the need for producers to produce numerous sizes of IOL to adjust them to numerous dimensions of eyes, which reduces the cost of production and storage which result. The ophthalmologists also benefit from IOL 12 in that they save time by eliminating the need to determine the dimension of the eye of each patient and the costs associated with maintaining large stocks of lenses of various sizes.

Another characteristic of the IOL 12 shown in the embodiment of FIG. 14 is one or more, but preferably between one and three, superficial edge grooves 78. The superficial edge grooves 78 permit more complete surgical irrigation and thus a better cleaning of the viscoelastic residues and the crystalline lens cortex residues within the crystalline lens capsule 18. During surgical irrigation, there is caused a fluid to circulate within the crystalline lens capsule 18 to remove the viscoelastic residues and various other residues. The superficial edge grooves 78 improve the circulation of the fluid by providing a clear path for the passage of the fluid. There can thus be carried out a more complete cleaning within the crystalline lens capsule 18 with the help of this reinforced circulation of fluid.

Although specific embodiments of the present invention have been shown and described above, it will be evident to those skilled in the art that various modifications can be adopted without departing from the spirit and scope of the concept of the invention and that the claims are not limited to the particular shapes shown and described herein.

1. Intraocular lens for implantation within an eye generally perpendicular to the optical axis of the eye, comprising:
an anterior surface (34);
a posterior surface (64);
an external peripheral edge 36 between said anterior and posterior surfaces defining an optical portion 34 with at least one portion of said external peripheral edge 36 and of said anterior surface forming an obtuse angle and at least one portion of said external peripheral edge 36 and of said posterior surface forming an acute angle;
two or more haptic elements 38 of the same transverse trapezoidal or rhomboidal cross section permanently connected to said external peripheral edge 36; and
acute external posterior haptic edges 52 on said haptic elements 38.

2. Intraocular lens for implantation in a crystalline lens capsule generally perpendicular to the optical axis of the eye to prevent posterior capsular opacification, comprising:
an anterior surface (34);
a posterior surface (64);
an external peripheral edge 36 between said anterior surface (34) and said posterior surface (64) defining an optical portion 34 with at least one portion of said external peripheral edge 36 and of said posterior surface forming an acute angle;
two or more haptic elements 38 of the same transverse trapezoidal or rhomboidal cross section permanently connected to said external peripheral edge 36; and
acute external posterior haptic edges 52 on said haptic elements 38.

3. Intraocular lens according to claim 1 or 2, in which the haptic elements 38 and the optical portion 34 are both formed of a pliable or compressible material.

4. Intraocular lens according to claim 1 or 2, in which said lens is formed from a material selected from the group comprising silicone polymers, hydrocarbon and fluorinated hydrocarbon polymers, hydrogels, soft acrylic polymers, polyesters, polyamides, polyurethane, silicone polymers with hydrophilic monomer additions, polysiloxane elastomers containing fluorine and their combinations.

5. Intraocular lens according to claim 1 or 2, in which said lens is formed of a hydrophilic acrylic material.

6. Intraocular lens according to claim 1 or 2, in which said lens is formed of a hydrophilic acrylic material which contains between 17 and 27 percent by weight of water.

7. Intraocular lens according to claim 1 or 2, in which said lens is formed of poly(HEMA-co-HOHEXMA).

8. Intraocuar lens according to claim 1 or 2, in which said lens is formed of one or several materials with at least one material having an index of refraction greater than 1.33.

9. Intraocular lens according to claim 1 or 2, in which said lens is formed of one or several materials with at least one material which is an acrylic material.

10. Intraocular lens according to claim 1 or 2, in which said lens is formed of one or several materials with at least one material which is a silicone material.

11. Intraocular lens according to claim 1 or 2, in which said haptic elements 38 have a homogenous thickness.

12. Intraocular lens according to claim 1 or 2, in which a zone of reduced brightness 74 is formed adjacent the external peripheral edges 36 of the optical portion 34.

13. Process for production of intraocular lenses according to claim 1 or 2, comprising:
forming a disk of one or several suitable materials, and machining said lenses from said disk.

14. Intraocular lens to be implanted within an eye, in the posterior chamber of said eye, perpendicular to the optical axis of said eye, comprising:
an anterior surface;
a posterior surface;
an external peripheral edge 36 between said anterior surface and said posterior surface defining an optical portion 34 with at least one portion of said external peripheral edge 36 and of said posterior surface forming an acute angle;
two or more haptic elements 38 of the same transverse trapezoidal or rhomboidal cross section permanently connected to said external peripheral edge 36; and
acute external posterior haptic edges 52 on said haptic elements 38.

15. Intraocular lens according to claim 1 or 2, in which said external peripheral edge 36 and said posterior surface define an acute angle between said haptic elements 38.

16. Intraocular lens according to claim 1 or 2, in which said external peripheral edge 36 and said anterior surface define an obtuse angle between said haptic elements 38.

17. Intraocular lens according to any one of the preceding claims in which the haptic elements (38) have a trapezoidal cross section so as to have an acute angle (52) which blocks the migration of the capsular cells along the posterior surface (64) of the optical portion (34).

18. Intraocular lens according to any one of the preceding claims in which the haptic elements (38) have a rhomboidal cross section so as to have an acute angle (52) which blocks the migration of the capsular cells along the posterior surface (64) of the optical portion (34).

19. Intraocular lens according to one or the other of claims 17 and 18 in which the trapezoidal or rhomboidal shape of the haptic elements (38) promotes the phenomenon of symphysis, which is to say the adherence of one against the other of the walls (30) and (32) of the capsular sac, which completely stops the migration of the capsular cells.
